(19) United States
(12) Patent Application Publication
(10) Pub. No.: US 2008/0140204 A1
(43) Pub. Date: Jun. 12, 2008

(54) Vertebral Implant Systems and Methods of Use

(75) Inventor: Eric S. Heinz, Memphis, TN (US)

Correspondence Address: COATS & BENNETT/MEDTRONICS, 1400 CRESCENT GREEN, SUITE 300, CARY, NC, 27518, US

(73) Assignee: Warsaw Orthopedic, Inc., Warsaw, IN (US)

(21) Appl. No.: 11/567,876

(22) Filed: Dec. 7, 2006

(51) Int. Cl. A61F 2/44

(52) U.S. Cl. 623/17.16; 623/17.15

(57) ABSTRACT

The present application is directed to vertebral implant systems and methods of using the systems. In one embodiment, the vertebral implant system includes first and second implants each including opposing end plates that are independently adjustable to form an included angle. Each implant within the system may include a different included angle to fit within a different sized and/or shaped vertebral space. Tool engaging features may be positioned on each end plate. The features on the opposing end plates may be substantially parallel when the implant is oriented at its specific included angle. The parallel orientation provides for accurate positioning of the implant within the vertebral space and may also assist in attaching and detaching the implant from the insertion tool. Using the system may include determining an anatomical angle of the vertebral space. Once the angle is determined, one of the implants from the system is selected that best matches the anatomical angle. The selected implant is then positioned with the contact features being substantially parallel. The implant may then be engaged with the insertion tool and inserted into the vertebral space.

### BACKGROUND

Intervertebral spinal implants are often used in the surgical treatment of spinal disorders such as degenerative disc disease, disc herniations, fractures, scoliosis, and other curvature abnormalities. Many different types of treatments are used. In some cases, spinal fusion is indicated to inhibit relative motion between vertebral bodies. In other cases, dynamic implants are used to preserve motion between vertebral bodies. Further, various types of implants may be used, including intervertebral and interspinous implants. Other implants are attached to the exterior of the vertebrae, whether at a posterior, anterior, or lateral surface of the vertebrae.

A normal human spine includes natural posterior and anterior curves (kyphotic and lordotic). The curvature exists because most vertebral bodies are not perfectly parallel to one another. Instead, there is usually a slight angle between vertebral bodies that, when compounded with angles between other vertebral bodies, results in the spinal curvature. In certain types of vertebral implants, the end plates of the implant are placed in contact with the vertebral bodies. Therefore, the end plates may also assume a non-parallel orientation. Further, for implants inserted at different vertebral levels along the spine, the end plates may assume different relative angles.

Since the implant end plates are generally angled relative to one another, insertion of the implant becomes difficult. Insertion is further complicated by the fact that the insertion direction is generally not parallel to either of the non-parallel end plates. As a result, insertion tools used to implant the non-parallel end plates tend to be complex and/or suited to a specific implant to be inserted at a particular vertebral level.

### SUMMARY

Illustrative embodiments disclosed herein are directed to vertebral implant systems and methods of using the systems. In one embodiment, the vertebral implant system includes first and second implants each including opposing end plates that are independently adjustable to form an included angle to accommodate insertion within a vertebral space. Each implant within the system may include a different included angle to fit within a different sized and/or shaped vertebral space. Tool engaging features may be positioned on each end plate. The features on the opposing end plates may be substantially parallel when the implant is oriented at its specific included angle. The parallel orientation provides for accurate positioning of the implant within the vertebral space and may also assist in attaching and detaching the implant from the insertion tool.

One method of using a vertebral implant system may include determining an anatomical angle of a vertebral space formed within a spine. Once the angle is determined, one of the implants from the system is selected that best matches the anatomical angle. The selected implant is then positioned with the contact features being substantially parallel. The implant may then be engaged with the insertion tool and inserted into the vertebral space.

### BRIEF DESCRIPTION OF THE DRAWINGS

FIG. 1 is a schematic view of a plurality of implants, an insertion tool, and a vertebral space according to one embodiment.

FIG. 2 is a sectional view of an implant according to one embodiment.

FIG. 3 is a sectional view of an implant according to one embodiment.

FIG. 4A is a perspective view of an implant according to one embodiment.

FIG. 4B is a sectional view cut along line IV-IV of FIG. 4A.

FIG. 5 is a partial schematic view of an insertion tool according to one embodiment.

FIG. 6 is a schematic view of an implant with engagement features aligned generally parallel with an insertion direction according to one embodiment.

FIG. 7 is a schematic view of an implant with engagement features aligned generally across an insertion direction according to one embodiment.

### DETAILED DESCRIPTION

The present application is directed to vertebral implant systems and methods of replacing a vertebral member. The system includes implants that include a pair of opposing engaging features for attachment with an insertion tool. Each of the implants is designed to form an included angle. The engaging features are in a parallel orientation when the implants are at their designed included angle. The parallel orientation provides for each of the implants to be engaged with a single insertion tool. The parallel orientation also provides for easier engagement with the insertion tool for installation and removal from the tool after insertion into the vertebral space.

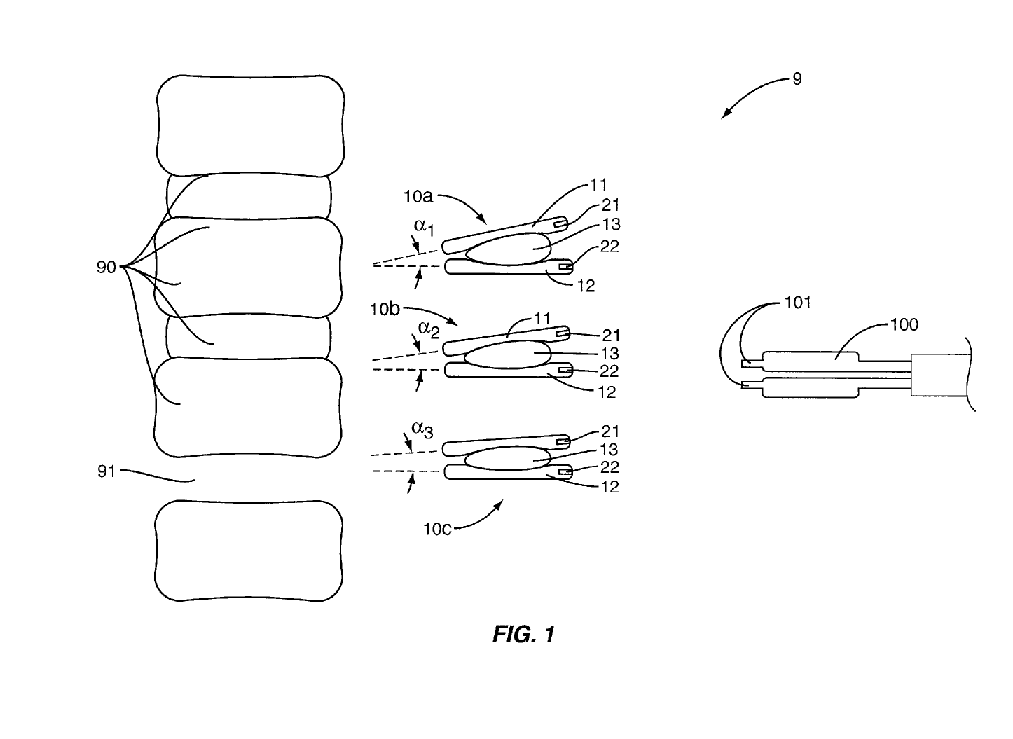

FIG. 1 schematically illustrates a plurality of implants 10a, 10b, 10c collectively referred to as element 10. The implants 10 include a superior end plate 11, an inferior end plate 12, and an intermediate section 13. Engagement features 21, 22 are positioned on each end plate 11, 12 to engage with an insertion tool 100 for inserting and positioning the implant 10 into the spine. The implants 10 are sized to fit within a vertebral space 91 formed within the spine. The vertebral space 91 may be formed along the spine by removal of one or more vertebral members 90. The vertebral members 90 may include vertebrae or intervertebral discs, or portions thereof.

Each of the superior and inferior end plates 11, 12 are positioned to form an included angle α1, α2, α3. Each implant 10a, 10b, 10c includes a different included angle α1, α2, α3 with implant 10a including a first included angle α1, implant 10b a second included angle α2, and implant 10c a third included angle α3. In one embodiment, the implants 10 are motion preserving devices that allow for movement of the vertebral members 90 after insertion of the implant 10. As such, one or both of the end plates 11, 12 are movable about the intermediate section 13. The engagement features 21, 22 are positioned to be substantially parallel when the included angle is at a predetermined size. The parallel orientation of the engagement features 21, 22 provides for a single insertion tool to engage each different implant 10. The parallel orientation also provides for easier attachment and removal of the implants 10 to and from the insertion tool 100.

A method of use may include initially determining an anatomical angle of the vertebral space 91. One of the implants 10 with a corresponding included angle α1, α2, α3 that best fits the anatomical angle is then selected to fit within the vertebral space 92. The engagement features 21, 22 of the selected implant 10 are placed in a parallel orientation to mount with the insertion tool 100. Once mounted, the implant 10 is placed within the vertebral space 91 while the implant 10 is at the corresponding included angle α. The implant 10 may be held by the tool 100 at this angle α while being attached to the vertebral members 90. In this manner, the tool 100 provides for accurately positioning the implant 10. The parallel orientation of the engagement features 21, 22 also provides for detachment of the implant 10 from the tool 100 after insertion.

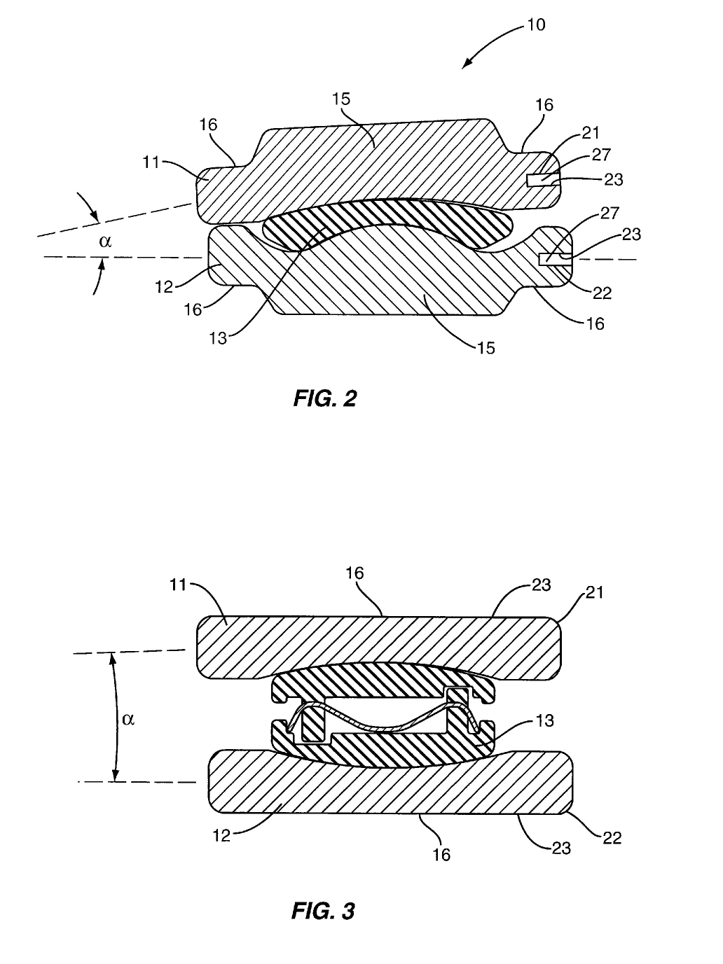

FIGS. 2 and 3 each illustrate embodiments of a vertebral implant 10 comprising three main components: a first end plate 11, a second end plate 12, and an intermediate section 13. The end plates 11, 12 include contact surfaces 16 to contact the vertebral members 90 that border on the vertebral space 91. The contact surfaces 16 may include various shapes and dimensions to match the configuration of the vertebral members 90. The contact surface 16 of the superior and inferior end plates 11, 12 may be the same or different. In one embodiment, each contact surface 16 is concave. As illustrated in FIG. 2, one or both end plates 11, 12 may also include a keel 15 that extends outward to maintain the implant 10 within the vertebral space 92.

The intermediate member 13 is positioned between the end plates 11, 12. Intermediate member 13 includes an arcuate shape that provides for relative movement between with one or both end plates 11, 12. Further, the inner surfaces of the end plates 11, 12 and outer surfaces of the intermediate member 13 may be polished to a fine surface finish and the spherical radii of the bearing surfaces may be the same or substantially similar. This provides for pivoting or sliding motion that changes the included angle α. The intermediate member 13 may be a single piece as illustrated in FIG. 2, or multiple pieces as illustrated in FIG. 3.

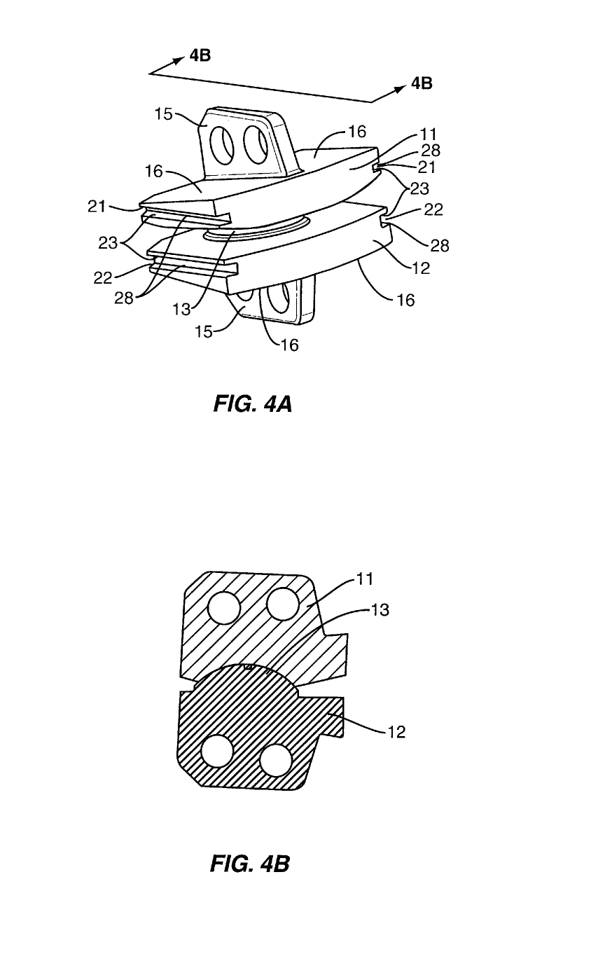

FIGS. 4A and 4B illustrate a two-piece construction. Implant 10 includes the intermediate member 13 being integrally formed with the end plate 12. The intermediate member 13 is formed by a combination of a curved receptacle in the superior end plate 11 and a semi-spherical extension on the inferior end plate 12. Contact surfaces 16 are positioned on each end plate 11, 12 to contact the vertebral members 90. Keels 15 extend outward from each of the end plates 11, 12.

Examples of implants are disclosed in U.S. Pat. Nos. 6,740,118 and 5,562,738, U.S. patent application Publication No. 2005/0038515, and U.S. patent application Ser. No. 11/343,954 entitled “Intervertebral Spinal Implant Devices and Methods of Use” filed on Jan. 31, 2006, each herein incorporated by reference.

Engagement features 21, 22 are positioned on the end plates 11, 12 to engage with an insertion tool 100. Each engagement features 21, 22 include an engagement surface 23 that is contacted by the insertion tool 100. The engagement surfaces 23 are positioned to receive a compressive force from the insertion tool 100 to maintain attachment. In one embodiment, the engagement surfaces 23 are substantially parallel when the end plates 11, 12 are positioned at their desired included angle α.

Engagement features 21, 22 may include a number of different configurations. FIG. 2 includes engagement features 21, 22 comprising receptacles 27 that extend into the end plates 11, 12. FIG. 3 includes the engagement features 21, 22 formed by the outer surface of the end plates 11, 12. These outer surfaces may be a section of the contact surfaces 16 that contact the vertebral members 90, or may be separate areas on the outer surfaces. The engagement features 22, 23 of FIGS. 4A and 4B include slots 28 that extend along the sides of the end plates 11, 12.

In these embodiments, the engagement feature 22 in the superior end plate 11 is substantially the same as the feature 23 in the inferior end plate 12. In other embodiments, the features 22, 23 in the end plates 11, 12 may be different. By way of example, end plate 11 includes a slot 28 and end plate 12 includes an outer surface. Further, the various shapes and sizes of the features 22, 23 may vary on each end plate 11, 12.

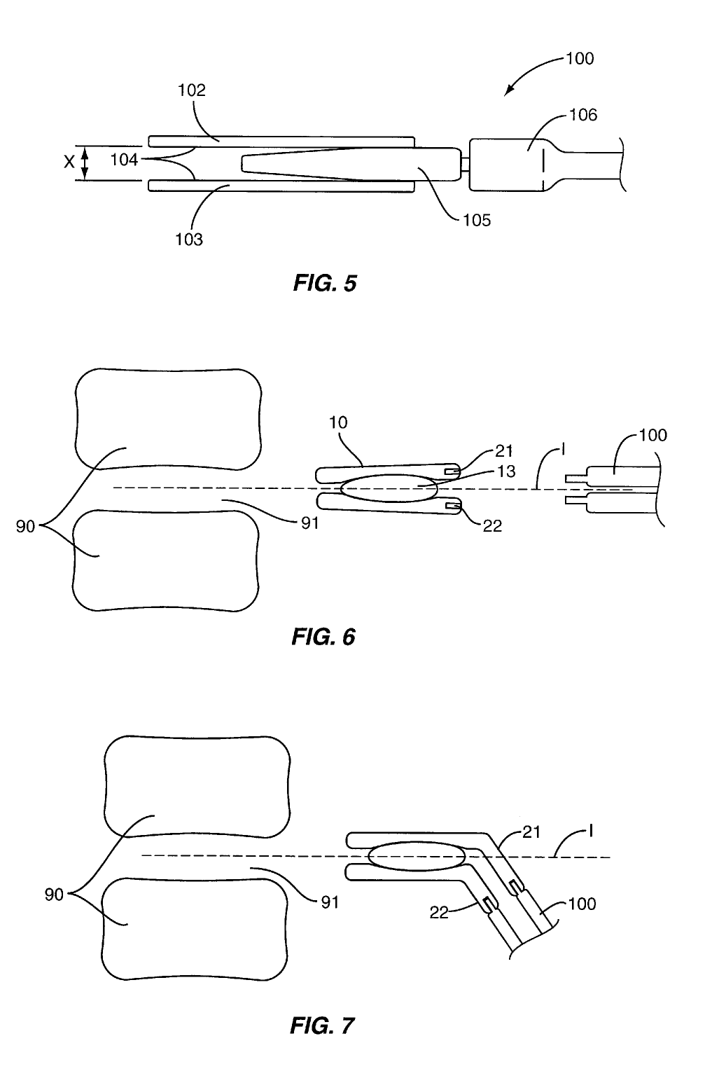

The insertion tool 100 engages the implants 10 for insertion and placement into the vertebral space 91. FIG. 5 illustrates one embodiment of a tool 100 that includes a first member 102 and a second member 103. Each of the members 102, 103 includes a surface 104 that contacts the implant 10. The surfaces 104 are positioned in a parallel arrangement when in contact with the implant 10. The members 102, 103 are movable in the direction of arrow X to adjust a distance between the contact surfaces 104. An adjustment member 105 may be positioned adjacent to the members 102, 103 to control the movement of the members 102, 103 and the distance between the contact surfaces 104.

In use, the tool 100 is oriented with members 102, 103 placed a distance apart and positioned at the engagement features of the implant 10. The members 102, 103 are then moved together to engaged the surfaces 104 with the contact surfaces 23 on the implant 10. The members 102, 103 apply a compressive force to maintain attachment with the implant 10. Once attached, the insertion tool 100 is manipulated by the surgeon to place the implant 10 within the vertebral space 91. The implant 10 remains at the desired included angle α while attached to the insertion tool 100. The insertion tool 100 may be used for accurate placement of the implant 10. After the implant 10 is positioned and/or mounted within the vertebral space 91, the members 102, 103 are moved outward. The outward movement removes the compressive force for detaching the implant 10 from the tool 100. One embodiment of an insertion tool is disclosed in U.S. patent application Ser. No. 11/549,661 entitled “Surgical Tool for Insertion of Spinal Prosthesis” filed on Oct. 16, 2006, hereby incorporated by reference.

The engagement features 21, 22 may be positioned for engagement with the insertion tool 100 in a variety of angular orientations. FIG. 6 illustrates the engagement features 21, 22 generally aligned in a parallel manner along the direction of insertion I. This alignment allows for the tool 100 to be moved in the direction of insertion I when engaging the implant 10. In another embodiment as illustrated in FIG. 7, the engagement features 21, 22 are aligned in a non-parallel manner with the direction of insertion I. The tool 100 engages the implant 10 while moving across the insertion direction I.

The vertebral members 90 forming the vertebral space 91 may require some amount of surgical preparation to accept the implant 10. This may include contouring to match the shape and contours end plates 11, 12 and/or bone removal to create recesses for inserting the keels 22.

In another embodiment, engagement features 22, 23 may include an extension that extends outward from the end plates 11, 12. These extensions include an engagement surface 23 and fit within receptacles formed within the insertion tool 100.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. In one embodiment, the implant 10 replaces a section of a vertebral member 90. In another embodiment, the implant 10 supplements the existing vertebral members 90 and does not replace a removed vertebral member 90. The present embodiments are, therefore, to be considered in all respects as illustrative and not restrictive, and all changes coming within the meaning and equivalency range of the appended claims are intended to be embraced therein.

What is claimed is:

1. A vertebral implant system comprising:
first and second implants each including opposing end plates that are independently adjustable to form an included angle, each of the end plates including a tool engaging feature to receive an insertion tool during insertion of the implants between the vertebral members;
the tool engaging features of the first implant being substantially parallel when their corresponding end plates are oriented to form a first predetermined included angle for insertion within a vertebral space of a first size, and the tool engaging features of the second implant being substantially parallel when their corresponding end plates are oriented to form a second predetermined included angle for insertion within a second vertebral space of a second size.

2. The system of claim 1, wherein the tool engaging features on the first and second implants are different.

3. The system of claim 1, wherein the tool engaging features on the first implant are different.

4. The system of claim 1, wherein at least one of the tool engaging features includes a receptacle formed within the end plate.

5. The system of claim 1, wherein at least one of the tool engaging features includes an extension that extends outward from the end plate.

6. The system of claim 1, wherein at least one of the tool engaging features includes a contact surface positioned on an outer side of the end plate.

7. The system of claim 1, wherein at least one of the tool engaging features includes a slot that extends along a lateral side of the implant, the slot formed by first and second rails that are spaced apart.

8. The system of claim 1, wherein the tool engaging features of the first implant are arranged in an overlapping configuration.

9. A vertebral implant system comprising:
first and second implants each including end plates with opposing faces that form an included angle to accommodate insertion between vertebral members, each of the end plates including an associated contact surface to contact an insertion tool;
the contact surfaces of the first implant being substantially parallel when their corresponding faces are oriented to form a first predetermined included angle for insertion within a first vertebral space with a first size, and the contact surfaces of the second implant being substantially parallel when their corresponding faces are oriented to form a second predetermined included angle for insertion within a second vertebral space with a second size.

10. The system of claim 9, wherein at least one of the contact surfaces is positioned within a receptacle that extends into one of the end plates.

11. The system of claim 9, wherein at least one of the contact surfaces is positioned on an extension that extends outward from one of the end plates.

12. The system of claim 9, wherein at least one of the contact surfaces is positioned on an outer edge of one of the opposing faces.

13. The system of claim 9, wherein the first implant includes different first and second contact surfaces.

14. A vertebral implant system comprising:
first and second implants each including a pair of associated contact surfaces to contact an insertion tool during insertion within the vertebral space;
the contact surfaces of the first implant being substantially parallel when the first implant is positioned to form a predetermined first included angle for insertion within the vertebral space, and the contact surfaces of the second implant being substantially parallel when the second implant is positioned to form a second included angle for insertion within the vertebral space.

15. The system of claim 14, wherein each of the first and second implants includes a pair of opposing end plates and an intermediate member.

16. The system of claim 14, wherein the contact surfaces of the first and second implants are positioned in an overlapping configuration.

17. The system of claim 14, wherein the contact surfaces of the first implant are different.

18. A vertebral implant system comprising:
a first implant including first and second end plates independently moveable to adjust a first included angle formed between the first and second end plates, the first end plate comprising a first contact surface and the second end plate comprising a second contact surface; and
a second implant including third and fourth end plates independently moveable to adjust a second included angle formed between the third and fourth end plates, the third end plate comprising a third contact surface and the fourth end plate comprising a fourth contact surface;
the first and second contact surfaces being substantially parallel when the first and second end plates are oriented to form a predetermined first included angle, and the third and fourth contact surfaces being substantially parallel when the third and fourth end plates are oriented to form a predetermined second included angle.

19. A method of using a vertebral implant system comprising:
preparing a plurality of implants, each implant including movable superior and inferior faces that form a different included angle;
determining an anatomical angle of a vertebral space formed within a spine;
selecting one of the plurality of implants with a corresponding included angle that best matches the anatomical angle;
engaging an insertion tool when engagement features associated with each of the superior and inferior faces of the selected implant are substantially parallel, the inferior and superior faces of the selected implant being at the corresponding included angle when the engagement features are substantially parallel; and
inserting the selected implant into the vertebral space.

20. The method of claim 19, wherein the step of engaging the insertion tool with each of the superior and inferior faces of the selected implant comprises engaging contact surfaces positioned on exterior surfaces of the end plates.

21. The method of claim 19, wherein the step of engaging the insertion tool with each of the superior and inferior faces of the selected implant comprises inserting contact features within receptacles that extend into the end plates.

22. The method of claim 19, further comprising engaging the insertion tool with each of the superior and inferior faces of the selected implant at an angle that is substantially parallel with an insertion direction.

23. The method of claim 19, further comprising engaging the insertion tool with each of the superior and inferior faces of the selected implant at an acute angle relative to an insertion direction.

24. The method of claim 19, further comprising engaging the insertion tool with lateral sides of the each of the end plates.

25. A method of using a vertebral implant system comprising:
determining an anatomical angle of a vertebral space formed within a spine;
selecting one of a plurality of implants with a corresponding included angle that best matches the anatomical angle;
positioning contact surfaces of the selected implant in a substantially parallel orientation to form the corresponding included angle and then engaging an insertion tool;
inserting the selected implant into the vertebral space with the insertion tool;
maintaining the selected implant at the corresponding included angle and attaching the implant within the spine; and
detaching the insertion tool from the selected implant.
